(19) United States
(12) Patent Application Publication
(10) Pub. No.: US 2005/0226822 A1
(43) Pub. Date: Oct. 13, 2005

(54) Oral care products containing ovomucin

(75) Inventors: Christine Garbers, Lorrach (DE); Karin Beatrice Merck, Oss (NL); Gijsbertus Anthonius Kleter, At Wageningen (NL); Johannes Maria Vereijken, Bennekom (NL); Gerardus Ferdinandus Johannes Rasing, Ad Wageningen (NL)

Correspondence Address: LEYDIG VOIT & MAYER, LTD, 700 THIRTEENTH ST. NW, SUITE 300, WASHINGTON, DC, 20005-3960, US

(73) Assignee: GABA INTERNATIONAL AG, MUNCHENSTEIN (CH)

(21) Appl. No.: 10/512,309

(22) PCT Filed: Apr. 25, 2003
(86) PCT No.: PCT/CH03/00271

§ 371 Date: May 16, 2005

(30) Foreign Application Priority Data
Apr. 25, 2002 (CH) 707/02

(51) Int. Cl. A61K 7/28

(52) U.S. Cl. 424/50

(57) ABSTRACT

The invention relates to oral care products, especially saliva substitution fluids, containing a mucus agent and an emulsifier. The invention also relates to oral care products containing a combination of mannoprotein and a mucus agent containing an ovomucin.

The present invention relates to oral care compositions. In particular, it relates to those oral care compositions in the form of saliva substitute fluids.

With respect to humectant action on the oral mucous membranes, oral care compositions customarily contain a humectant, e.g. polyhydric alcohols such as glycerol, propylene glycol, sorbitol, mannitol, glucose syrup; polyethylene glycols, polypropylene glycols; polyvinyl-pyrrolidone; or cellulose derivatives such as, for example, hydroxyethylcellulose.

Oral care compositions in the form of saliva substitute fluids are employed in the case of an inadequate natural flow of saliva. The abnormal inadequate flow of saliva designated in technical language as “xerostomial” causes the affected person, for example, to have trouble in speaking, chewing and swallowing. While speaking, he must often take a sip of water or a drink in order to restore the moistness of the oral cavity, but this only has a short effect. Xerostomics are susceptible to infections of the oral cavity, in particular also of the salivary ducts.

In a saliva substitute fluid, not only must the humectant effect on the oral mucous membranes be achieved, but the correct rheological behavior of the saliva substitute fluid must preferably also be achieved. Human saliva is a thixotropic liquid, its viscosity decreases with increasing shear stress. This property is important for its action as a lubricant in articulation and in chewing. The thixotropy of the saliva is caused by glycoproteins (mucins) contained therein. The mucin which occurs in human saliva typically contains 50 to 90% by weight of carbohydrates and approximately 3.9 mg/100 g of sialic acid and has a molecular weight of typically approximately 200 to approximately 1000 kDa.

Saliva substitute fluids containing different humectants and/or mucous agents are obtainable on the market. Examples of these are (the active agent contained therein in brackets): “Glandosane” from Cell Pharm GmbH, Germany (cellulose), “Xialine” from Lommerse Pharma, Holland (xanthan resin), “Stoppers 4 Dry Mouth” from Woodridge Labs, USA (glycerol and hydroxyethylcellulose), “Oralbalance” from Laclède International, Belgium (starch and hydroxyethyl-cellulose), and “Saliva medac” from Medac Gesellschaft für klinische Spezialpräparate, Germany (mucin from pigs' stomachs). In the case of saliva substitute fluids which contain mucins of animal origin as mucous agent, the acceptance of the patients is questionable, which springs from the idea that it can be offputting to take a preparation into the mouth which, for example, originates from the mucous membrane of a pig's stomach.

The mucin ovomucin has been known as a substance (better as a substance mixture) for a long time (e.g. Robinson, D. S., Monsey, J. B., The Biochemical Journal 1966, 100/2, 61ff., and literature cited therein). It occurs in various constituents of eggs, in particular the egg white. Typically, the soluble fraction of the egg white, salts, and other low molecular weight compounds are dissolved out of the raw egg white (previously separated from the chalaza and from the egg membranes by sieving) first by diluting in several volumes of water and soluble proteins. The thick fraction of the egg white remaining as a gel is washed with further water and/or with KCl solution, the ovomucin precipitating. This ovomucin contains subunits which are designated as α- and β-ovomucin. α-Ovomucin contains N-glycosidically bonded carbohydrates (about 15% by weight) and only a little sialic acid. β-Ovomucin contains O-glycosidically bonded carbohydrates (about 50 to 60% by weight) and a lot of sialic acid. Besides the actual ovomucin glycoprotein, often further non-mucin proteins are also present, in particular also lysozyme (whose separation from the actual ovomucin is difficult, since it interacts with the latter). Ovomucin is thus not a pure substance, but a substance mixture which is not precisely characterizable, in which the actual ovomucin glycoprotein occurs more or less dominantly.

The isolated ovomucin is initially very poorly soluble or not soluble in water. The reason lies in the oxidation of thiol groups of the cysteine present in ovomucin with formation of disulfide groups. In this process, crosslinkages between the individual mucin chains are formed which lead to extremely high molecular weight polymers. The solubilization of the ovomucin after isolation is as a rule brought about by reductants such as mercaptoethanol (back-reduction of the disulfide bridges to thiols) and alternatively by additional use of thiol-blocking agents such as HgCl₂, mercury bis(p-chlorobenzoate) or iodoacetic acid. These agents, however, are all toxic. It is also known that the solubility of ovomucin increases with increasing NaCl content of the solution (up to approximately 2 M) (Rabouille, C., Aon, M. A., Thomas, D., Archives of Biochemistry and Biophysics, 1989, 270(2), 495-503). Such salts contents, however, are not utilizable in oral care compositions.

The rheological properties of ovomucin solutions, alternatively also in combination with a single further additive, selected from lysozyme, NaCl or CaCl₂, have been investigated (Hayakawa, S., Sato, Y., Agric. Biol. Chem. 1978, 42(11), 2025-2029).

It is known that in egg white a complex between ovomucin and the non-mucin protein lysozyme is present (inter alia Miller, S. M., Kato, A., Nakai, S., J. Agric. Food Chem. 1982, 30, 1127-1132).

In WO-A-99/04804, oral care compositions, for example saliva substitute fluids, are described which can contain, inter alia, deovalbumized egg white and/or aqueous extract of egg yolk. WO-A-99/04804 stresses the significance of the immunologically active constituents of the egg, e.g. lysozyme and ovotransferrin, in the oral care compositions.

It is known of some of the proteins present in the egg white, e.g. of ovalbumin, lysozyme and ovotransferrin, that they can act as allergens.

It is the object of the present invention to make available oral care compositions having good humectant action on the oral mucous membrane, which are more favorable in the acceptance to the patients than the previously known oral care compositions, in which mucins of animal origin provide for humectant action, and which are accompanied by a decreased danger of allergic reactions. In particular, it is the object of the present invention to make available those improved saliva substitute fluids which also preferably approach the rheological properties of human saliva.

The object is achieved by oral care compositions which comprise:
a) a mucous agent which contains ovomucin and has a sialic acid content of 1.5 to 2.5 percent by weight, and
b) an emulsifier.

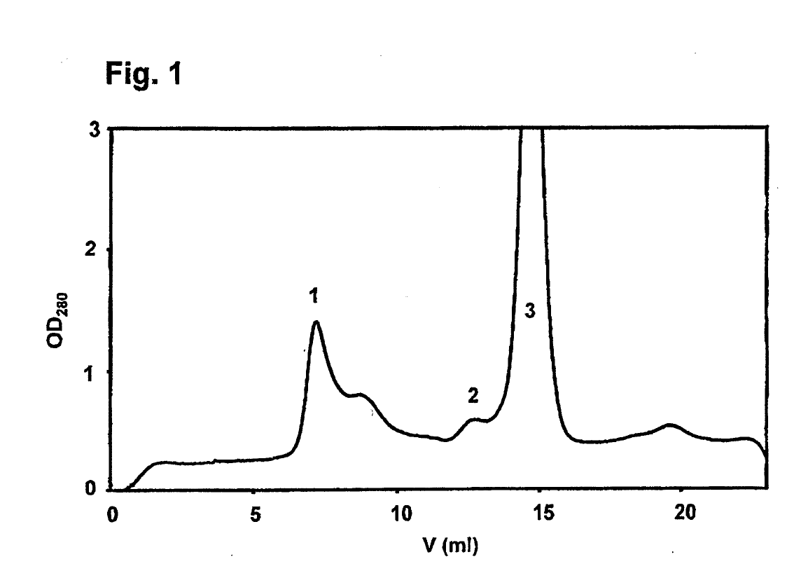

FIG. 1 is a chromatographic separation of a) an ovomucin-containing mucous agent which can be used according to the invention, as prepared in example 1. The conditions of the chromatography are according to example 2. X-axis: eluted volume, in ml; Y-axis: optical density of the eluate, measured at 280 nm. Identified compounds: 1 ovomucin; 2 ovalbumin (43 kDa) and ovotransferrin (77 kDa); 3 lysozyme (14 kDa).

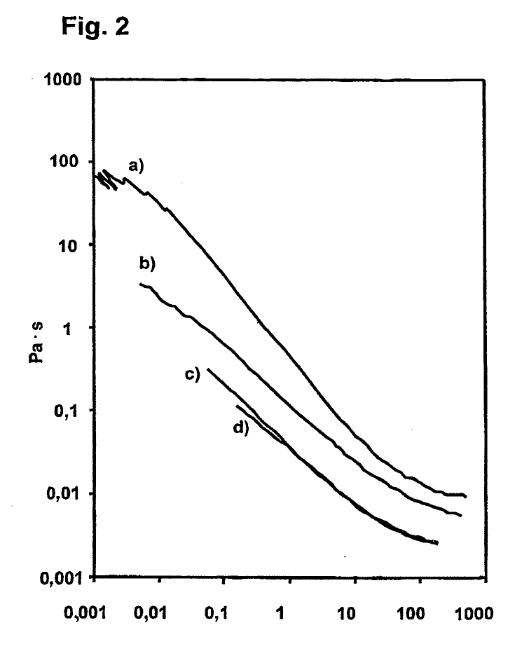

FIG. 2 is a representation of the rheological behavior of various mucous agents at 37° C. X-axis: shear rate in rotations per second; Y-axis: measured viscosity in Pascal×seconds. The following denote: a) dried sample of an ovomucin-containing mucous agent, as prepared in example 1, b) mucin from pigs' stomachs, obtained from Sigma, c) and d) two different samples of mucin from pigs' stomachs, obtained from ICN.

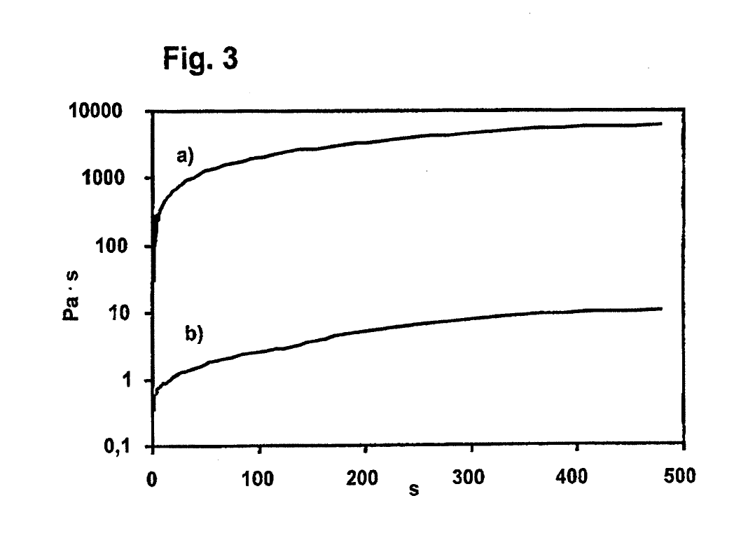

FIG. 3 shows the time course of the restoration of the viscosity at 37° C. after high shear stress. X-axis: timescale in seconds, Y-axis: viscosity in Pascal×seconds. a) is the ovomucin-containing mucous agent from example 1, and b) is a mucin from pigs' stomachs (obtained from Sigma).

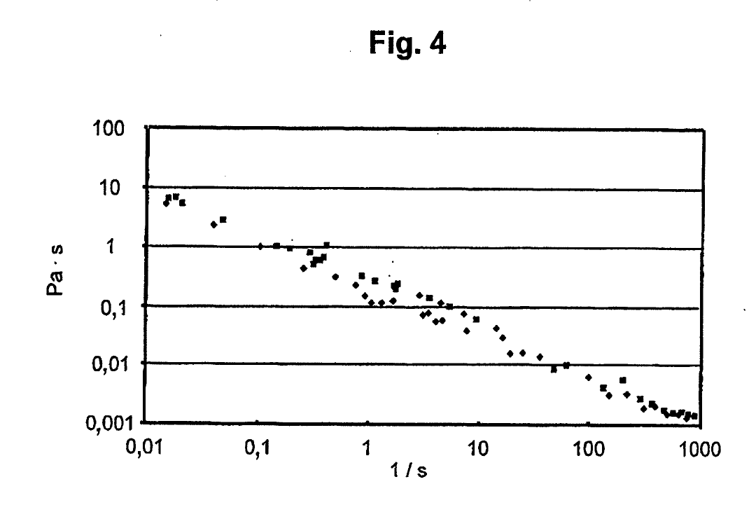

FIG. 4 is a representation of the rheological behavior at 37° C. of two samples (rhombi or squares) of a saliva substitute fluid (just under 2 percent by weight dry matter of ovomucin-containing mucous agent, based on the weight of the fluid) containing 0.15 percent by weight of Cremophor® RH 410 as an emulsifier. X-axis: shear rate in rotations per second; Y-axis: measured viscosity in Pascal×seconds.

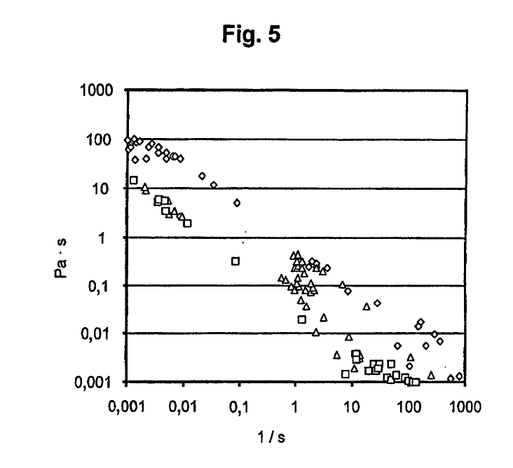

FIG. 5 is a representation of the rheological behavior at 37° C. of three samples (triangles, rhombi, squares) of a saliva substitute fluid (just under 2 percent by weight dry matter of ovomucin, based on the weight of the fluid) containing 0.15 percent by weight of Tween 20®. X-axis: shear rate in rotations per second; Y-axis: measured viscosity in Pascal×seconds.

It was surprisingly found that by the use of a mucous agent which contains ovomucin or essentially consists of this, oral care compositions are obtained which effectively keep the oral mucous membrane moist. It was also found that when using such a mucous agent in oral care compositions in the form of a saliva substitute fluid a utilizable rheological behavior of such a saliva substitute fluid according to the invention is simultaneously obtained. It was also found that the solubility decrease of the ovomucin contained in the mucous agent to be employed according to the invention, which is caused by the extensive removal of the possibly allergenic proteins ovalbumin, lysozyme and ovotransferrin, can be compensated by addition of an emulsifier. It was additionally found in the case of the saliva substitute fluids according to the invention that the addition of the emulsifier does not disadvantageously influence the rheological behavior of the ovomucin contained in the mucous agent, i.e. the thixotropic behavior of the ovomucin is essentially maintained unchanged. The term “mucous agent” in the context of the present application means a viscosity-modifying additive for oral care compositions.

“Oral care composition” in the context of the present application is understood as meaning any oral care composition in which a mucous agent is customarily present, and in which according to the invention an ovomucin-containing mucous agent can be used instead of this mucous agent (or additionally to this), where the remaining additives and/or active substances can be chosen according to type and amount in analogy to the respective previously known oral care compositions. Examples of oral care compositions according to the invention are rinse solutions, touch-up solutions, and in particular saliva substitute fluids.

Rinse solutions according to the invention are preferably aqueous or alcoholic, particularly preferably mixed, aqueous/alcoholic solutions. They can typically contain a relatively dilute concentration of an active substance, e.g. of a customary disinfecting agent such as, for example, chlorhexidine or triclosan. Typical additives for rinse solutions, beside the ovomucin employed as a humectant agent and the active agents, are, for example, sweeteners such as saccharin, quaternary ammonium saccharinates, cyclamates, or aromatic substances such as, for example, coumarin and vanillin or ethereal oils, e.g. peppermint oil, spearmint oil, anisole, menthol, anethole, citrus oil, etc., or other aromas such as apple, eucalyptus or spearmint aroma.

Touch-up solutions according to the invention can be similar to the rinse solutions, but contain, in analogy to the previously known touch-up solutions, a relatively high amount of active agent.

Saliva substitute fluids according to the invention can be aqueous solutions which contain the ovomucin-containing mucous agent and various salts for adjusting the tonicity, the osmolarity and the pH in such a way that a chemical composition similar to human saliva results. Such additives are chlorides of the alkali metals or alkaline earth metals, (for example NaCl or MgCl₂) for adjusting the tonicity, and buffer substances such as, for example, phosphate or carbonate buffers, which adjust the pH of the saliva substitute fluid to a pH which, although it is physiologically tolerable in the oral cavity, is sufficiently high to counteract the dissolution of the dental enamel. The saliva substitute fluids according to the invention analogously also contain such additives.

Saliva substitute fluids according to the invention accordingly preferably contain 0.05 to 0.15 percent by weight, particularly preferably approximately 0.12 percent by weight, of KCl; preferably 0.05 to 0.1, particularly preferably approximately 0.086, percent by weight of NaCl; preferably 0.002 to 0.008, particularly preferably approximately 0.005, percent by weight of MgCl₂; preferably 0.01 to 0.02, particularly preferably approximately 0.015, percent by weight of CaCl₂; and they are preferably buffered with dihydrogenphosphate/hydrogenphosphate and/or carbonate/hydrogencarbonate buffer to a pH and a buffer capacity which corresponds to human saliva. Preferably, the saliva substitute fluids according to the invention can contain calcium salts in combination with phosphate in such a way that the dissolution of the dental enamel is likewise counteracted.

The oral care compositions according to the invention contain only those amounts of ovalbumin, lysozyme and ovotransferrin which are present as an unavoidable impurity in the ovomucin-containing mucous agent isolated from egg white. In the preparation of the oral care compositions according to the invention, no other additives selected from ovalbumin, lysozyme or ovotransferrin or containing these are used.

The ovomucin-containing mucous agent to be employed according to the invention is on the one hand the precipitate which can be obtained from egg white (for example and preferably from egg white of hens' eggs) by diluting with a number of, typically 2 to 10, volumes of water, allowing this solution to stand for the purpose of precipitation at typically 0° C. to approximately room temperature and subsequent removal by centrifugation. The precipitation from the aqueous solution of egg white can be favored, for example, by adjusting the pH of the solution to approximately the isoelectric point of the ovomucin glycoprotein contained in ovomucin (i.e. to approximately 4.0 to 6.0, preferably to approximately 5.0 to approximately 6.0). Slightly oxidizing conditions (e.g. atmospheric oxygen) can also be chosen, which involve the oxidation of the thiol groups of the ovomucin glycoproteins to give disulfide bridges (intra- and intermolecular), whereby a reduction in the solubility of the ovomucin is likewise achieved. These two variants are reversible (by shifting the pH from the isoelectric region or by re-reduction of the disulfide bridges by means of, for example, excess 2-mercaptoethanol).

Ovomucin-containing mucous agents which can be employed according to the invention can also be isolated from other constituents of eggs, in particular from the chalazae and the egg yolk membranes. These constituents are essentially an ovomucin of higher degree of polymerization than the ovomucin from egg white. In particular, the ovomucin from the chalazae and the egg yolk membranes is very similar to the ovomucin from egg white with respect to the chemical composition, for example the sialic acid content. For isolation of a mucous agent which can be used according to the invention from the chalazae or egg yolk membranes, these can previously be centrifuged for the separation of possible solids (remains of egg shells), if necessary, and subsequently subjected to washing with, in turn, typically 2 to 10 volumes of water. In the preparation of an oral care composition according to the invention, advantageously a dispersion of the ovomucin-containing mucous agent which is as good as possible is brought about (e.g. by mixing under high shear action, for example with an Ultraturrax® mixer), and possible insoluble remains can advantageously be separated off by centrifugation, sedimentation or filtration.

On washing with typically 2 to 10 volumes of water, a major part of the non-mucin, possibly allergenic proteins (e.g. ovalbumin, ovotransferrin, lysozyme) is removed from the mucous agent, since these are more soluble than ovomucin. This is manifested in the fact that the content of sialic acid (expressed in grams of sialic acid per 100 grams of dry matter, measurable by colorimetric test using acidic ninhydride reagent, see Yao, K., Ubuka, T., Masuoka, N., Kinuta, M., Ikeda, T., Anal. Biochem. 1989, 179, 332-335, this publication is included by way of reference) increases greatly compared with the content in the original egg white. The ovomucin-containing mucous agent thus isolated then typically contains approximately 1.5 to approximately 2.5 grams of sialic acid per 100 grams of dry matter, preferably approximately 1.7 to approximately 2.0 grams per 100 grams of dry matter. If, following the literature, a content of approximately 2 percent by weight of sialic acid is assumed for pure ovomucin, it can be assumed therefrom that the mucous agent thus isolated consists essentially, that is to at least approximately 80 percent by weight of the dry matter, presumably even to more than 90 percent by weight, of ovomucin.

If desired, the ovomucin-containing mucous agent can, as generally known, be purified further by further washing with water and/or KCl solutions. In the examples and figures, an ovomucin-containing mucous agent from egg white further purified in this way was investigated. According to the knowledge of the inventor, further washing, however, brings about no further significant change in the sialic acid content or the ratio of the extinctions at 280 nm and at 214 nm (this is a measure of the residual content, in particular of lysozyme). This shows that, even on first diluting the egg white with typically 2 to 10 volumes of water, allowing to stand and centrifuging, by far the largest part of the foreign proteins and salts are removed. According to the invention, it is therefore thus not absolutely necessary to perform a continuing purification of the ovomucin-containing mucous agent.

It is presumed that the residual content of lysozyme present in the mucous agent and the nature and amount of the salts typical for an oral care composition, in particular for a saliva substitute fluid, increase the solubility of the ovomucin contained in the mucous agent in the oral care compositions according to the invention. The solubility of the ovomucin can, if necessary, also be increased by nontoxic reducing agents for disulfide bridges such as, for example, cysteine, reducing vitamin C derivatives or NADPH. The mucous agent can be digested with these agents before use in the oral care compositions according to the invention or in favorable cases suitable amounts of the agents can be added directly as additives.

The solubility of the ovomucin-containing mucous agent is increased by emulsifiers, such as are customarily used for the dispersion of poorly water-soluble aromatic substances in oral care compositions (in particular synthetic emulsifiers). Examples of such emulsifiers are anionic emulsifiers (e.g. alkali metal salts of fatty acids or of fatty alcohol sulfates, such as, for example, sodium lauryl sulfate) or zwitterionic emulsifiers. Examples of the latter are the amides of (C₁₀-C₂₀)fatty acids with N′,N′-dialkyl-N′-carboxy-methyl-(C₂-C₄) diaminoalkylene as an amide-forming radical, the two amino groups of the diaminoalkylene being terminal. A particularly preferred example of this is cocamidopropylbetaine. Nonionic emulsifiers are also preferred such as, for example, the poly(oxyethylene) derivatives of a partially esterified (C₃-C₆)sugar alcohol, the acids esterifying the sugar alcohol being (C₁₀-C₂₀)fatty acids, which can alternatively be hydroxylated on the hydrocarbon chain. Among the fatty acids hydroxylated on the hydrocarbon chain, ricinoleic acid is preferred. “Partially” esterified means, as customary in chemistry, that at least one hydroxyl group of the sugar alcohol is not esterified. The sugar alcohol is preferably glycerol, mannitol or sorbitol. The number of oxyethylene units in the poly(oxyethylene) derivative can preferably be in the range from 5 to 50, preferably approximately 20 to approximately 40. The poly(oxyethylene) radical is, as customary in the art, obtained by reacting the partially esterified sugar alcohol with ethylene oxide. Particularly preferred examples of emulsifiers in the form of poly(oxyethylene) derivatives are the emulsifiers marketed under the trade names Cremophor® (e.g. Cremophor RH 410) and Tween® (e.g. Tween 20).

The amounts of the emulsifiers can preferably be employed in the mass ratio of ovomucin-containing mucous agent to emulsifier in the range from approximately 10:1 to approximately 1:2, where the quantitative ratio can be determined from the desired degree of dispersion of the ovomucin and/or from the possible simultaneous presence of poorly water-soluble flavorings.

The contents of neutral carbohydrates can be determined in the ovomucin-containing mucous agent to be employed according to the invention, if desired, according to previously known processes. The carbohydrates can be determined by acidic hydrolysis (TAPPI process T 249 cm-85) and determination of the corresponding alditol acetates by means of liquid/gas chromatography (Teunissen, W., Gosselink, R. J. A., Vezinhet, F.). Ovomucin which can be employed according to the invention can typically contain 2 to 40 percent by weight of neutral carbohydrates, preferably approximately 20 to 35 percent by weight of carbohydrates. The contents of carbohydrates are in this case approximately identical, like the corresponding average contents of approximately 33% in ovomucin according to the literature (Robinson, D. S., Monsey, J. B., The Biochemical Journal 1966, 100/2, 61-62).

The total content of nitrogen in the ovomucin-containing mucous agent to be employed according to the invention can, as customary, be determined according to Kjeldahl. It can typically contain approximately 11 to 14 percent by weight of nitrogen, preferably approximately 12 to 14 percent by weight of nitrogen. The total content of nitrogen can be converted into a total content of proteins by means of an empirical factor of 6.25.

Preferably, the oral care compositions according to the invention in the form of saliva substitute fluids have a similar thixotropy to human saliva. The concept of thixotropy here has the customary meaning, i.e. it designates the decrease in the dynamic viscosity η (in Pa.s) with increasing shear rate D (in s⁻¹) at a given, constant temperature.

In the context of the present application, the expression “thixotropy similar to human saliva” can mean that the quotient of the dynamic viscosity η of a saliva substitute fluid according to the invention and the dynamic viscosity η of human saliva for each value of the shear rate D in the range from 60 to 300 s⁻¹ always lies between approximately 0.1 and approximately 10, preferably always between approximately 0.3 and approximately 3, all measurements being performed at 37° C. The range of the shear rate indicated is typical for the shear actions which occur within the salivary layer of the oral mucous membranes on speaking and chewing.

At 37° C., human saliva itself typically has the rheological properties listed in Table 1 (mixed saliva sample from 11 human subjects, collection time 1400 hours). The measuring conditions here are the same as in the section “Measurements” from example 3.

TABLE 1

| Shear stress D (s⁻¹) | Viscosity η (Pa · s) |
|---|---|
| 46.961 | 0.002818 |
| 54.794 | 0.002787 |
| 63.861 | 0.002733 |
| 74.183 | 0.002597 |
| 85.880 | 0.002552 |
| 98.995 | 0.002467 |
| 113.76 | 0.002421 |
| 130.35 | 0.002340 |
| 148.86 | 0.002292 |
| 169.45 | 0.002249 |
| 192.46 | 0.002210 |
| 218.13 | 0.002199 |
| 246.58 | 0.002157 |
| 277.87 | 0.002090 |
| 312.71 | 0.002078 |
| 350.91 | 0.002081 |
| 393.07 | 0.002070 |

The values in Table 1 can be used as a reference curve for the above expression “thixotropy similar to human saliva”.

Since most of the additives mentioned at the outset influence the viscosity and the thixotropy of a saliva substitute fluid, it is preferred if initially the nature and amount of the additives are determined as they also occur in human saliva, and only then by means of a series of experiments is it determined what the proportion of ovomucin-containing mucous agent must be in order to achieve a similar thixotropy to human saliva. The content of ovomucin-containing mucous agent in the preferred oral care compositions according to the invention in the form of saliva substitute fluids can then typically be in the order of magnitude of approximately 0.01 to approximately 2 percent by weight.

Beside the ovomucin-containing mucous agent, further mucous agents, such as are already previously known (e.g. carboxymethylcellulose, hydroxyalkylcelluloses, water-soluble and swellable salts of the polyacrylic acids, alginates, carraghenates, guar gum, high molecular weight polyethylene oxide, animal mucins), can alternatively be added to the oral care compositions according to the invention. The amounts of these additional further mucous agents can typically be approximately 0.5 to approximately 5 percent by weight, based on the finished oral care composition, where the viscosity-modifying action of such an additional mucous agent can be taken into account.

For example, in the case of a saliva substitute fluid, ovomucin-containing mucous agent according to the invention in combination with a further mucous agent can be used in order to adjust the thixotropy as accurately as possible to the thixotropy of the human saliva. By the superposition of the Theological properties of the ovomucin with the Theological properties of the additional mucous agent used in combination, the thixotropic properties of the preferred saliva substitute fluids according to the invention can under certain circumstances be tuned more finely than only with ovomucin alone. The additional mucous agents which can be used in combination with the ovomucin-containing mucous agent can in this case produce (Newtonian) solutions which are thixotropic or nonthixotropic per se.

Particularly preferably, such oral care compositions according to the invention, in particular saliva substitute fluids, contain a combination of ovomucin-containing mucous agents with mannoprotein as an additional mucous agent. Mannoprotein is the generic term for a class of polysaccharides which are an important constituent of the cell walls. Examples of mannoproteins are the adhesins in Candida albicans and the agglutinins in Saccharomyces cerevisiae. These are in some cases commercially obtainable as partly purified, dried preparations and can be used according to the invention directly in this form. The mannoproteins from Saccharomyces cerevisiae are preferred. With respect to the obtainment and the properties of mannoproteins from Saccharomyces cerevisiae, reference can also be made to the literature (e.g. Barriga, J. A. T., Cooper, D. G., Idziak, E. S., Cameron, D. R., Enzyme and Microbial Technology 1999, 25, 96-102).

The weight ratio of ovomucin-containing mucous agent to mannoprotein in these oral care compositions according to the invention can be approximately 1:1000 to approximately 10:1, and preferably it is 1:100 to approximately 2:1. Ovomucin-containing mucous agent and mannoprotein are preferably employed in such oral care compositions according to the invention, in particular in saliva substitute fluids according to the invention, in amounts such that contents (in percentages by weight) of ovomucin-containing mucous agent of approximately 0.01 to approximately 5%, and of manno-protein of approximately 0.5 to approximately 10% result. Particularly preferably, ovomucin-containing mucous agent and mannoprotein are employed in amounts which result in approximately 0.05 to approximately 1.0% of ovomucin-containing mucous agent and approximately 2.0 to approximately to approximately 0.5% of mannoprotein.

In oral care compositions containing a combination of ovomucin-containing mucous agent and mannoprotein, the methods mentioned further above can also be employed for improving the solubility of the ovomucin, in particular also the emulsifiers mentioned there. Preferably, here too the ovomucin-containing mucous agent has a sialic acid content of 1.5 to 2.5 percent by weight, based on the dry matter.

Further humectants as additives can in all cases preferably also be added to the oral care compositions according to the invention. Such humectants are, for example, polyhydric alcohols such as glycerol, propylene glycol, sorbitol, mannitol, glucose syrup, polyethylene glycols or polypropylene glycols. If they are used, their amounts can typically be approximately 0.5 to approximately 5 percent by weight, based on the finished saliva substitute fluid.

Flavorings such as, for example, saccharin, quaternary ammonium saccharinates, cyclamates, coumarin, vanillin, and aromatic substances such as, for example, peppermint oil, spearmint oil, anisole, menthol, anethole, citrus oil etc., or other aromas such as apple, eucalyptus or spearmint aroma can preferably also be added as additives to the oral care compositions according to the invention. They can typically be added in amounts of approximately 0.5 to approximately 2 percent by weight, based on the finished oral care composition, where the strength and nature of the taste can be taken into account.

The oral care compositions according to the invention can also alternatively contain physiologically tolerable preservatives such as, for example, sodium benzoate or sorbic acid in antimicrobially active amounts as additives.

If an increased bactericidal or caries-preventative action is desired, one or more sources of fluoride ions can be added to the oral care compositions according to the invention. Examples of these are water-soluble inorganic fluoride salts such as NaF, KF and SnF₂, and organic ammonium fluorides such as Olaflur (N′-octadecyl-N′,N,N-tris(hydroxyethyl)-1,3-propanediamine dihydrofluoride) or the bis(2-hydroxyethyl)alkyl-ammonium fluorides described in the international patent publication WO-A-98/22427. These fluoride sources can be employed individually or in combination. The amounts of fluoride source can typically be chosen such that the finished oral care composition contains approximately 100 to approximately 1500 ppm of free fluoride.

The oral care compositions according to the invention can preferably be prepared by mixing of an ovomucin-containing mucous agent, in particular of such a mucous agent having a sialic acid content of 1.5 to 2.5 percent by weight, with an aqueous solution comprising an emulsifier. A particularly preferred process for the preparation of the oral care compositions according to the invention, in particular in the form of saliva substitute fluids, rinse solutions or touch-up solutions, comprises the following steps: a) The mucous agent isolated from egg white or chalazae, having a sialic acid content of approximately 1.5 to approximately 2.5 percent by weight of the dry matter, is suspended in an aqueous solution which contains the emulsifier and the possible salts typical for the oral care composition according to nature and amount, b) the suspension is stirred (e.g. briefly a number of times, approximately 3 to 10 times for 5 to 10 seconds each, with breaks in stirring of 10 to 20 seconds between two stirring operations), under high shear forces, preferably by means of a stirrer working according to the stator-rotor principle (for example an Ultraturrax®), if desired with cooling, c) the suspension is treated, if desired, with ultrasound (e.g. for a number of minutes, approximately 1 to 10 minutes, using, for example, an ultrasonic bath or an ultrasonic probe, if desired with cooling), d) if undissolved residues of the ovomucin-containing mucous agent remain, the oral care composition can preferably be stirred until the establishment of solution equilibrium (e.g. 1 to 5 hours at approximately 20 to 30° C., preferably at approximately room temperature) The undissolved residues of the mucous agent then still remaining can, if desired, be filtered off.

The oral care compositions according to the invention can be employed in analogy to the previously known saliva substitute fluids and, depending on the nature of the oral care composition, using the customary applicators such as, for example, customary bottles, brushes, squeeze bottles or alternatively, as spray solution using a suitable propellant such as, for example, CO₂. In the latter case, small spray bottles can be used as applicators.

The oral care compositions according to the invention can, if desired, be prepared only immediately before use from a solid formulation which contains the ovomucin-containing mucous agent, the alternative further additives and possible further mucous agents, and which, for example, is present in the form of a powder, a tablet or a pastille, by dissolving and diluting in a suitable amount of water and possible further solvents such as, for example, alcohol. These can be desirable if the finished oral care composition is not storable for a sufficient length of time. Such dry preparations for the production of an oral care composition according to the invention are a further object of the invention.

The invention is now illustrated further by the following examples. These serve only for illustration, but not for interpretation of the scope of protection.

### EXAMPLE 1

### Isolation of Ovomucin-Containing Mucous Agent from Hens' Egg White

Freshly removed egg white from hens' eggs was dissolved in three times the volume of distilled water and the pH of the solution was adjusted to 6 using citric acid.

The solution obtained was stirred at 4° C. for 20 hours. The solution was then centrifuged at 5500 g for 30 min at 4° C. and the centrifugation residue was washed twice with distilled water, twice with 2% by weight KCl in water and twice with distilled water, the wash solution being centrifuged at 5500 g for 30 min at 4° C.

The content of dry matter of this preparation was determined by drying a weighed sample under normal pressure for 16 hours at 75° C. and subsequently for 3 hours at 105° C. For the further tests, the ovomucin was used in non-dried form, assuming the dry matter content thus determined.

For longer storage of the preparation, this was stored at −20° C.

### EXAMPLE 2

### Chromatographic Separation of Ovomucin-Containing Mucous Agent

The separation was carried out following known processes (Awadé, A. C., Moreau, S., Mollé, D., Brulé, G., Maubois, J. L., J. Chrom. A. 1994, 677, 279-288). A solution was prepared by dissolving an amount of ovomucin-containing mucous agent from preparation example 1, which corresponded to 5 mg of dry matter, in 10 ml of water, and this solution was allowed to stand at 20° C. for 20 hours. 500 μl of the solution were separated by means of gel permeation chromatography (separation according to molecular weight) on a Superose 6 column (Amersham Pharmacia Biotech, separable MW range 5 to 5000 kDa). The mobile phase was 50 mM imidazole, 0.2 percent by volume of 2-mercapto-ethanol, 0.5 percent by weight of Na dodecyl sulfate (SDS) in water at pH 7, the elution rate was 0.45 ml/min. The eluted fractions were detected by means of measurement of the UV absorption at 280 nm. A chromatogram as shown in FIG. 1 was obtained. Since the ovomucin contained in the mucous agent is eluted in the dead volume (peak 1), it is to be assumed that it has a molecular weight of at least 5000 kDa.

### EXAMPLE 3

### Measurement of Rheological Properties of Solutions of Ovomucin-Containing Mucous Agent

Sample Preparation

A sample of the ovomucin-containing mucous agent from example 1 was dissolved in PBS buffer (0.2 g/l of KH₂PO₄, 1.441 g/l of Na₂HPO₄×2H₂O, 8.5 g/l of NaCl, 0.1 g/of MgCl₂×6H₂O, 0.2 g/l of KCl; pH 7) by means of an Ultraturrax and with gentle stirring. The initial weight of the ovomucin sample was such that a solution of 2 percent by weight (based on the dry matter of the mucous agent) resulted. The finished solution was stored at 4° C. overnight before the measurements.

Measurement

The rheological measurements were carried out on a dynamic stress rheometer SR-200 (Rheometric Scientific Inc., Piscataway, USA) at 37° C. A chromium-coated Couette cell of 16 ml capacity was used. Before the actual measurement, the sample was subjected to pretreatment with a shear stress of approximately 100 s⁻¹ for one minute and a subsequent recovery phase of 3 min.

For the measurement of the viscosity as a function of the shear stress (FIGS. 1 and 2), the test was begun at the lowest possible stress which the measuring apparatus was able to produce and the mechanical action was increased up to 50 Pa. The shear stress and the viscosity were measured.

For the measurement of the restoration of the viscosity (FIG. 3), the sample was subjected to the highest possible shear stress which the measuring apparatus was able to produce and subsequently the time course of the viscosity was recorded at the lowest possible shear stress which the measuring apparatus was able to produce.

### EXAMPLE 4

### Formulations for Oral Care Compositions According to the Invention

The general preparation procedure for the following examples 4a to 4j was as follows:

The oral care compositions of these examples (100 g each) were prepared from up to 6 different aqueous solutions A, B, C, D, E and F.

Solution A (if employed) contained the preservatives. For the preparation of solution A, the preservatives were weighed out in those amounts as indicated in the table of the respective example, and dissolved in 10 g of purified water at 50° C.

Solution B was prepared by dissolving the amount of the ovomucin-containing mucous agent indicated in the respective table, as prepared in example 1, in 20 g of purified water at a maximum of 30° C.

Solution C (if employed) contained possible additional humectants and mucous agents (in addition to ovomucin-containing mucous agent) . For this, the corresponding amounts of these compositions, as indicated in the respective table, were weighed out and dissolved in 15 g of purified water with warming to a maximum of 30° C.

Solution D contained the aromatic substances (if employed) and the emulsifiers possibly necessary. For this, the emulsifiers were first weighed out in those amounts as indicated in the respective table, and dissolved in 10 g of water. Subsequently, the amount of aromatic substances weighed out, as indicated in the respective table, was added and the mixture was dissolved with stirring.

Solution E contained further additives, such as, for example, buffer salts, sweeteners (artificial and/or natural), colorants, agents for the regulation of the osmotic pressure and the tonicity. For this, the additives, as indicated in the respective table, were weighed out in the corresponding amounts and dissolved in 10 g of water.

Solution F was a stock solution of the alternative fluoride ion donor NaF or Olaflur in water. In the case of NaF the concentration of stock solution was 50 mg of NaF/100 ml of solution and in the case of Olaflur it was 500 mg/100 ml of solution. In the preparation of the oral care compositions, if a fluoride ion source was desired, an aliquot of NaF or Olaflur stock solution was additionally used.

For the preparation of the finished oral care composition, solution B was first introduced. With stirring at room temperature, solution A was added (if employed), then solution C (if employed), then solution D (if employed), then solution E, then the remaining water needed for 100 g of oral care composition, if it had not already been used for the preparation of the other starting solutions to be used, and finally, if used, the abovementioned aliquot of stock solution F. After addition of each of the solutions, the mixture was stirred until homogenization was complete.

### EXAMPLE b 4a

### Saliva Substitute Fluid

| | Grams | Solution |
|---|---|---|
| Ovomucin-containing | 0.100 | B |
| mucous agent | | |
| Hydroxyethylcellulose | 2.000 | C |
| Methylparaben | 0.180 | A |
| Propylparaben | 0.020 | A |
| Aroma | 0.800 | D |
| Cremophor ® | 0.200 | D |
| K₂HPO₄ | 0.035 | E |
| KHCO₃ | 0.150 | E |
| KCl | 0.122 | E |
| NaCl | 0.086 | E |
| MgCl₂ | 0.005 | E |
| CaCl₂ | 0.015 | E |
| Sorbitol | 3.000 | E |
| Na saccharin | 0.100 | E |
| Water | to 100 | |

### EXAMPLE 4b

### Saliva Substitute Fluid

| | Grams | Solution |
|---|---|---|
| Ovomucin-containing | 0.100 | B |
| mucous agent | | |
| Mannoprotein | 3.000 | C |
| Methylparaben | 0.180 | A |
| Propylparaben | 0.020 | A |
| Aroma | 0.800 | D |
| Cremophor ® | 0.200 | D |
| K₂HPO₄ | 0.035 | E |
| KHCO₃ | 0.150 | E |
| KCl | 0.122 | E |
| NaCl | 0.086 | E |
| MgCl₂ | 0.005 | E |
| CaCl₂ | 0.015 | E |
| Na saccharin | 0.100 | E |
| Water | to 100 | |

### EXAMPLE 4c

### Saliva Substitute Fluid

| | Grams | Solution |
|---|---|---|
| Ovomucin-containing | 0.500 | B |
| mucous agent | | |
| Carboxymethylcellulose | 1.500 | C |
| Sodium benzoate | 0.200 | A |
| Sorbic acid | 0.100 | A |
| Tween ® 20 | 0.15 | D |
| K₂HPO₄ | 0.035 | E |
| KHCO₃ | 0.150 | E |
| KCl | 0.122 | E |
| NaCl | 0.086 | E |
| MgCl₂ | 0.005 | E |
| CaCl₂ | 0.015 | E |
| Xylitol | 2.000 | E |
| Saccharin | 0.200 | E |
| NaF | 0.0005 | F (aliquot of |
| | | 1 ml) |
| Water | to 100 | |

### EXAMPLE 4d

### Saliva Substitute Fluid

| | Grams | Solution |
|---|---|---|
| Ovomucin-containing | 0.500 | B |
| mucous agent | | |
| Mannoprotein | 5.000 | C |
| Methylparaben | 0.180 | A |
| Propylparaben | 0.020 | A |
| Aroma | 0.800 | D |
| Cremophor ® | 0.200 | D |
| K₂HPO₄ | 0.035 | E |
| KHCO₃ | 0.150 | E |
| KCl | 0.122 | E |
| NaCl | 0.086 | E |
| MgCl₂ | 0.005 | E |
| CaCl₂ | 0.015 | E |
| Saccharin | 0.200 | E |
| Water | to 100 | |

### EXAMPLE 4e

### Saliva Substitute Fluid

| | Grams | Solution |
|---|---|---|
| Ovomucin-containing | 0.750 | B |
| mucous agent | | |
| Hydroxyethylcellulose | 1.000 | C |
| Aroma | 0.700 | D |
| Tween ® 20 | 0.2 | D |
| Na₂HPO₄ | 0.028 | E |
| KHCO₃ | 0.150 | E |
| KCl | 0.120 | E |
| NaCl | 0.005 | E |
| MgCl₂ | 0.005 | E |
| CaCl₂ | 0.015 | E |
| Mannitol | 2.000 | E |
| Na saccharin | 0.200 | E |
| Water | to 100 | |

### EXAMPLE 4f

### Saliva Substitute Fluid

| | Grams | Solution |
|---|---|---|
| Ovomucin-containing | 0.750 | B |
| mucous agent | | |
| Mannoprotein | 2.000 | C |
| Sodium benzoate | 0.200 | A |
| Sorbic acid | 0.100 | A |
| Aroma | 0.700 | D |
| Cremophor ® | 0.200 | D |
| Na₂HPO₄ | 0.028 | E |
| KHCO₃ | 0.150 | E |
| KCl | 0.120 | E |
| NaCl | 0.005 | E |
| MgCl₂ | 0.005 | E |
| CaCl₂ | 0.015 | E |
| Na saccharin | 0.200 | E |
| Water | to 100 | |

### EXAMPLE 4g

### Saliva Substitute Fluid

| | Grams | Solution |
|---|---|---|
| Ovomucin-containing | 1.000 | B |
| mucous agent | | |
| Carboxymethylcellulose | 1.000 | C |
| K₂HPO₄ | 0.035 | E |
| KCl | 0.122 | E |
| KHCO₃ | 0.150 | E |
| NaCl | 0.086 | E |
| MgCl₂ | 0.005 | E |
| CaCl₂ | 0.015 | E |
| Xylitol | 3.000 | E |
| Saccharin | 0.200 | E |
| Aroma | 0.900 | D |
| Cremophor ® | 0.200 | D |
| Sodium benzoate | 0.200 | A |
| Sorbic acid | 0.100 | A |
| Water | to 100 | |

### EXAMPLE 4h

### Saliva Substitute Fluid

| | Grams | Solution |
|---|---|---|
| Ovomucin-containing | 1.000 | B |
| mucous agent | | |
| Mannoprotein | 2.000 | C |
| Methylparaben | 0.180 | A |
| Propylparaben | 0.020 | A |
| Aroma | 0.900 | D |
| K₂HPO₄ | 0.035 | E |
| KHCO₃ | 0.150 | E |
| KCl | 0.122 | E |
| NaCl | 0.086 | E |
| MgCl₂ | 0.005 | E |
| CaCl₂ | 0.015 | E |
| Saccharin | 0.200 | E |
| Olaflur | 0.0035 | F (aliquot of |
| | | 7 ml) |
| Water | to 100 | |

### EXAMPLE 4i

### Rinse Solution

| | Grams | Solution |
|---|---|---|
| Ovomucin-containing | 0.100 | B |
| mucous agent | | |
| Xylitol | 2.500 | C |
| Aroma | 0.100 | D |
| (peppermint/spearmint) | | |
| PEG 40-hydrogenated | 0.200 | D |
| castor oil (Cremophor ® | | |
| RH 410, BASF) | | |
| 0.4% strength pigment | 0.050 | E |
| solution of Ariavit | | |
| Blue 3.85 CI 42051 | | |
| Acesulfam K | 0.025 | E |
| Ethanol | 5.000 | E |
| Olaflur | 0.125 | F (aliquot of |
| | | 25 ml) |
| Water | to 100 | |

### EXAMPLE 4j

### Rinse Solution

| | Grams | Solution |
|---|---|---|
| Ovomucin-containing | 0.100 | B |
| mucous agent | | |
| Mannoprotein | 2.500 | C |
| Aroma | 0.100 | D |
| (peppermint/spearmint) | | |
| PEG 40-hydrogenated | 0.200 | D |
| castor oil (Cremophor ® | | |
| RH 410, BASF) | | |
| 0.4% strength pigment | 0.050 | E |
| solution of Ariavit | | |
| Blue 3.85 CI 42051 | | |
| Acesulfam K | 0.025 | E |
| Ethanol | 5.000 | E |
| Olaflur | 0.125 | F (aliquot of |
| | | 25 ml) |
| Water | to 100 | |

### EXAMPLE 4k

### Touch-Up Solution

| | Grams | Solution |
|---|---|---|
| Ovomucin-containing | 0.100 | B |
| mucous agent | | |
| Aroma (consisting of | 2.500 | D |
| 30 parts of anisole, | | |
| 7.5 parts of menthol, | | |
| 1.0 parts of vanillin, | | |
| 6.0 parts of spearmint | | |
| oil and 55.5 parts of | | |
| peppermint oil) | | |
| Tween ® 20 | 0.2 | D |
| Olaflur | 10.000 | Undissolved, |
| | | weighed in |
| | | solid. |
| Saccharin | 0.150 | E |
| Water | to 100 | |

In example 4k the same process description was followed as for examples 4a to 4j, except that first 10,000 g of Olaflur were weighed out and subsequently the solutions B, D, E and the remaining water, as indicated in the general process description, were added.

### EXAMPLE 4l

### Saliva Substitute Fluid

| | Grams | Solution |
|---|---|---|
| Ovomucin-containing | 0.100 | B |
| mucous agent | | |
| Hydroxyethylcellulose | 2.000 | C |
| Methylparaben | 0.180 | A |
| Propylparaben | 0.020 | A |
| Aroma | 0.800 | D |
| Tween ® 20 | 0.200 | D |
| K₂HPO₄ | 0.035 | E |
| KHCO₃ | 0.150 | E |
| KCl | 0.122 | E |
| NaCl | 0.086 | E |
| MgCl₂ | 0.005 | E |
| CaCl₂ | 0.015 | E |
| Sorbitol | 3.000 | E |
| Na saccharin | 0.100 | E |
| Water | to 100 | |

### EXAMPLE 4m

### Saliva Substitute Fluid

| | Grams | Solution |
|---|---|---|
| Ovomucin-containing | 0.100 | B |
| mucous agent | | |
| Mannoprotein | 3.000 | C |
| Methylparaben | 0.180 | A |
| Propylparaben | 0.020 | A |
| K₂HPO₄ | 0.035 | E |
| KHCO₃ | 0.150 | E |
| KCl | 0.122 | E |
| NaCl | 0.086 | E |
| MgCl₂ | 0.005 | E |
| CaCl₂ | 0.015 | E |
| Na saccharin | 0.100 | E |
| Water | to 100 | |

### EXAMPLE 4n

### Saliva Substitute Fluid

| | Grams | Solution |
|---|---|---|
| Ovomucin-containing | 0.500 | B |
| mucous agent | | |
| Mannoprotein | 5.000 | C |
| Methylparaben | 0.180 | A |
| Propylparaben | 0.020 | A |
| Aroma | 0.800 | D |
| Tween ® 20 | 0.200 | D |
| K₂HPO₄ | 0.035 | E |
| KHCO₃ | 0.150 | E |
| KCl | 0.122 | E |
| NaCl | 0.086 | E |
| MgCl₂ | 0.005 | E |
| CaCl₂ | 0.015 | E |
| Saccharin | 0.200 | E |
| Water | to 100 | |

### EXAMPLE 4o

### Saliva Substitute Fluid

| | Grams | Solution |
|---|---|---|
| Ovomucin-Containing | 0.750 | B |
| mucous agent | | |
| Mannoprotein | 2.000 | C |
| Sodium benzoate | 0.200 | A |
| Sorbic acid | 0.100 | A |
| Aroma | 0.700 | D |
| Tween ® 20 | 0.200 | D |
| Na₂HPO₄ | 0.028 | E |
| KHCO₃ | 0.150 | E |
| KCl | 0.120 | E |
| NaCl | 0.005 | E |
| MgCl₂ | 0.005 | E |
| CaCl₂ | 0.015 | E |
| Na saccharin | 0.200 | E |
| Water | to 100 | |

### EXAMPLE 4p

### Saliva Substitute Fluid

| | Grams | Solution |
|---|---|---|
| Ovomucin-containing | 1.000 | B |
| mucous agent | | |
| Carboxymethylcellulose | 1.000 | C |
| K₂HPO₄ | 0.035 | E |
| KCl | 0.122 | E |
| KHCO₃ | 0.150 | E |
| NaCl | 0.086 | E |
| MgCl₂ | 0.005 | E |
| CaCl₂ | 0.015 | E |
| Xylitol | 3.000 | E |
| Saccharin | 0.200 | E |
| Aroma | 0.900 | D |
| Tween ® 20 | 0.200 | D |
| Sodium benzoate | 0.200 | A |
| Sorbic acid | 0.100 | A |
| Water | to 100 | |

### EXAMPLE 4q

### Rinse Solution

| | Grams | Solution |
|---|---|---|
| Ovomucin-containing | 0.100 | B |
| mucous agent | | |
| Xylitol | 2.500 | C |
| Aroma | 0.100 | D |
| (peppermint/spearmint) | | |
| Tween ® 20 | 0.200 | D |
| 0.4% strength pigment | 0.050 | E |
| solution of Ariavit | | |
| Blue 3.85 CI 42051 | | |
| Acesulfam K | 0.025 | E |
| Ethanol | 5.000 | E |
| Olaflur | 0.125 | F (aliquot of |
| | | 25 ml) |
| Water | to 100 | |

### EXAMPLE 4r

### Rinse Solution

| | Grams | Solution |
|---|---|---|
| Ovomucin-containing | 0.100 | B |
| mucous agent | | |
| Mannoprotein | 2.500 | C |
| Aroma | 0.100 | D |
| (peppermint/spearmint) | | |
| Tween ® 20 | 0.200 | D |
| 0.4% strength pigment | 0.050 | E |
| solution of Ariavit | | |
| Blue 3.85 CI 42051 | | |
| Acesulfam K | 0.025 | E |
| Ethanol | 5.000 | E |
| Olaflur | 0.125 | F (aliquot of |
| | | 25 ml) |
| Water | to 100 | |

### EXAMPLE 5

### Measurement of Rheological Properties in a Saliva Substitute Fluid According to the Invention

Preparation of the Saliva Substitute Fluid

A sample of an ovomucin-containing mucous agent (as prepared in example 1) which contained 4 g of dry matter was suspended in 200 ml of PBS buffer (1.102 g/l of KH₂PO₄, 0.262 g/l of Na₂HPO₄×2H₂O, 0.585 g/l of NaCl, 0.1 g/ of MgCl₂×6H₂O, 0.2 g/l of KCl; pH 6.5). The suspension was first stirred with a stirrer working according to the stator-rotor principle (Ultraturrax IKA T25 basic, rod diameter 1.8 cm) 3 times every 20 seconds at 11,500 rpm with cooling with an ice bath, with a stirring break of 15 seconds between two stirring operations. The suspension was then sonicated with ultrasound by means of an ultrasonic probe (Branson Sonifier 450, rod diameter 1.2 cm) 20 times for 5 seconds each at setting 6 and with cooling in an ice bath, with a stirring break of 15 seconds between each sonication. 0.3 g of emulsifier (Cremophor® RH 410 or Tween® 20) was then added and the finished saliva substitute fluid was stirred at room temperature for 2 hours. Only the supernatant of the solution was used for the measurement; according to sialic acid content determination, at least approximately 80 percent by weight of the ovomucin-containing mucous agent employed was dissolved.

Measurement

The rheological measurements were carried out on a dynamic stress rheometer SR-200 (Rheometric Scientific Inc., Piscataway, USA) at 370° C. A 40 mm cell with chromium-coated parallel plates having a separation of 0.200 mm was used. Before the actual measurement, the sample of the saliva substitute fluid was subjected to pretreatment with a shear stress of approximately 100 s⁻¹ for one minute and a subsequent recovery phase of 3 min. For the actual measurement, the test was begun under the lowest possible stresses which the measuring apparatus was able to produce, and the stress was increased up to 50 Pa.

The shear stress and the viscosity of three samples of the saliva substitute fluid prepared and pretreated in this way was measured, and the behavior as shown in FIGS. 4 and 5 obtained.

1. An oral care composition comprising:
a) a first mucous agent which contains ovomucin and has a sialic acid content of 1.5 to 2.5 percent by weight, and
b) an emulsifier, wherein the oral care composition contains only those amounts of ovalbumin, lysozyme, and ovotransferrin which are present as an impurity in the first mucous agent.

2. An oral care composition comprising:
a) a mucous agent which contains ovomucin, and
b) mannoprotein.

3. The oral care composition as claimed in claim 2, wherein the weight ratio of mucous agent to mannoprotein is 1:1000 to 10:1.

4. The oral care composition as claimed in claim 1, comprising a second mucous agent which is selected from the group consisting of carboxymethyl-cellulose, hydroxyalkylcelluloses, water-soluble and swellable salts of polyacrylic acids, alginates, carraghenates, guar gum, high molecular weight polyethylene oxide, and animal mucins and mixtures thereof.

5. The oral care composition as claimed in claim 2, comprising an emulsifier.

6. The oral care composition as claimed in claim 1, wherein the emulsifier is a synthetic emulsifier.

7. The oral care composition as claimed in claim 6, wherein the emulsifier is a poly-(oxyethylene) derivative of a partially esterified (C₃-C₆)sugar alcohol, acids esterifying the sugar alcohol being (C₁₀-C₂₀)fatty acids, which are alternatively hydroxylated on the hydrocarbon chain.

8. The oral care composition as claimed in claim 7, wherein the poly(oxyethylene) derivative comprises 5 to 50 oxyethylene units.

9. The oral care composition as claimed in claim 6, wherein the emulsifier is zwitterionic.

10. The oral care composition as claimed in claim 1, wherein the oral care composition is a saliva substitute fluid.

11. The saliva substitute fluid as claimed in claim 10, comprising additives which are selected from the group consisting of NaCl, CaCl₂, MgCl₂, KCl, dihydrogen-phosphates and hydrogen phosphates of sodium or potassium, and carbonates and hydrogen carbonates of sodium or potassium.

12. The saliva substitute fluid as claimed in claim 11, wherein the additives are 0.05 to 0.15 percent by weight of KCl, 0.05 to 0.1 percent by weight of NaCl, 0.002 to 0.008 percent by weight of MgCl₂ and 0.01 to 0.02 percent by weight of CaCl₂, and the saliva substitute fluid is buffered with dihydrogen phosphate/hydrogen phosphate and/or carbonate/hydrogen carbonate buffer to a pH and a buffer capacity which corresponds to human saliva.

13. The saliva substitute fluid as claimed in claim 12, wherein the saliva substitute fluid contains approximately 0.12 percent by weight of KCl, approximately 0.086 percent by weight of NaCl, approximately 0.005 percent by weight of MgCl₂, and approximately 0.015 percent by weight of CaCl₂.

14. The saliva substitute fluid as claimed in claim 10, having a thixotropy similar to human saliva.

15. The oral care composition as claimed in claim 1, comprising a humectant as an additive.

16. The oral care composition as claimed in claim 15, wherein the humectant is selected from the group consisting of glycerol, propylene glycol, sorbitol, mannitol, glucose syrup, polyethylene glycols, and polypropylene glycols.

17. The oral care composition as claimed in claim 1, comprising a synthetic aroma as an additive.

18. The oral care composition as claimed in claim 1, comprising a source of fluoride ions as an additive.

19-20. (canceled)

21. A process for the preparation of an oral care composition, comprising mixing an ovomucin-containing mucous agent having a sialic acid content of 1.5 to 2.5 percent by weight, based on dry matter, with an aqueous solution which comprises an emulsifier, wherein the oral care composition lacks additives selected from ovalbumin, lysozyme, ovotransferrin, and additives containing them.

22. The process as claimed in claim 21, comprising:
a) suspending an ovomucin-containing mucous agent having a sialic acid content of 1.5 to 2.5 percent by weight, based on dry matter, in an aqueous solution which comprises an emulsifier,
b) stirring the suspension obtained in step a) under high shear forces,
c) optionally treating the suspension with ultrasound, if a suspension remains after step b), and
d) optionally stirring the suspension, if a suspension remains after step c).

23. The oral care composition as claimed in claim 1, in solid form

24. The oral care composition as claimed in claim 2, wherein the oral care composition is a saliva substitute fluid.

25. The saliva substitute fluid as claimed in claim 24, having a thixotropy similar to human saliva.

26. The oral care composition as claimed in claim 2, comprising a humectant as an additive.

27. The oral care composition as claimed in claim 2, comprising a synthetic aroma as an additive.

28. The oral care composition as claimed in claim 2 further comprising a source of fluoride ions as an additive.

29. The oral care composition as claimed in claim 2, in solid form
